(19) United States
(12) Patent Application Publication
(10) Pub. No.: US 2023/0010314 A1
(43) Pub. Date: Jan. 12, 2023

(54) HEAD INJURY ASSESSMENT BASED ON COMNINATIONS OF BIOMARKERS, COGNITIVE ASSESSMENT AND/OR IMPACT DETECTION

(71) Applicant: HitlQ Limited, South Melbourne (AU)

(72) Inventors: Michael Vegar, Queenscliff (AU); David Erikson, Queenscliff (AU)

(21) Appl. No.: 17/811,019

(22) Filed: Jul. 6, 2022

(30) Foreign Application Priority Data
Jul. 6, 2021 (AU) 2021902062

(51) Int. Cl. A61B 5/00; A61B 10/00; A61B 5/11; A61B 5/145; G16H 50/20

(52) U.S. Cl. CPC A61B 5/4064; A61B 5/682; A61B 10/0051; A61B 5/11; A61B 5/14507; A61B 5/7267; A61B 5/7275; G16H 50/20; A61B 2562/06; A61B 2503/10

(57) ABSTRACT

Improved assessment of brain injuries, and improved brain injury management, is achieved using a combination of impact-related data derived from instrumented mouthguard devices, human function performance testing, and biomarkers derived from biological fluid (such as saliva and/or blood). The human function performance testing may include brain function performance testing, and/or other forms of human function performance testing. This involves combining a data-driven understanding of a head impact event (based on data collected via an instrumented mouthguard device) with a data-driven understanding of human function performance following that head impact event. The biomarkers may include salivary mRNA and/or ncRNA, and/or blood proteins (for example via a FDA-approved Brain Trauma Indicator test).

### FIELD OF THE INVENTION

The present invention relates, in various embodiments, to head injury assessment based on combinations of biomarkers, cognitive assessment and/or impact detection. For example, some embodiments relate to a sideline head injury assessment technique for sportspersons. Other embodiments include AI classification technologies. While some embodiments will be described herein with particular reference to those applications, it will be appreciated that the invention is not limited to such a field of use, and is applicable in broader contexts.

### BACKGROUND

Any discussion of the background art throughout the specification should in no way be considered as an admission that such art is widely known or forms part of common general knowledge in the field.

Brain injuries, particularly those sustained during participation in contact sports, are becoming an increasingly important focus of attention. For example, head impacts sustained during sport can have serious effects of both short term and long-term participant welfare. For example, it is valuable to better understand the nature of a suspected brain injury in terms of: (i) whether a participant should be rested from participation; (ii) an extent to which the injury should prevent a return to activity; (iii) a degree of seriousness of an injury, for instance insofar as that might affect treatment and management; and (iv) better understanding cumulative effects of successive brain injuries for a given participant.

### SUMMARY OF THE INVENTION

It is an object of the present invention to overcome or ameliorate at least one of the disadvantages of the prior art, or to provide a useful alternative.

Example embodiments are described below in the section entitled “claims”.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

### BRIEF DESCRIPTION OF THE DRAWINGS

Embodiments of the invention will now be described, by way of example only, with reference to the accompanying drawings in which:

FIG. 1A to FIG. 10 illustrate an instrumented mouthguard in varying states of assembly.

FIGS. 2A and 2B illustrate an example PCB component for an instrumented mouthguard.

FIG. 3 illustrates a mouthguard device according to one embodiment.

FIG. 4 illustrates a system according to one embodiment example use cases for technology described herein.

FIG. 5 illustrates a VR technology framework according to one embodiment.

### DETAILED DESCRIPTION

The present invention relates, in various embodiments, to technology adapted for improved assessment of brain injuries in a human subject.

In overview, improved assessment of brain injuries, and improved brain injury management, is achieved using a combination of impact-related data derived from instrumented mouthguard devices, human function performance testing, and biomarkers derived from biological fluid (such as saliva and/or blood). The human function performance testing may include brain function performance testing, and/or other forms of human function performance testing. This involves combining a data-driven understanding of a head impact event (based on data collected via an instrumented mouthguard device) with a data-driven understanding of human function performance following that head impact event. The biomarkers may include salivary mRNA and/or ncRNA, and/or blood proteins (for example via a FDA-approved Brain Trauma Indicator test).

As described herein, “instrumented mouthguard devices” are devices which include sensor components (for example one or more accelerometers, and optionally one or more gyroscopes), which collect data from which head impacts are able to be detected and quantified. The process of detection and quantification varies between embodiments, and various approaches are known in the art. A known approach includes translating linear and/or rotational acceleration measurements detected by mouthguard sensors to a defined location on the human head, and quantifying impact parameters based on accelerations at that point. Some embodiments make use of models, for example Finite Element Analysis models, thereby to model potential brain injuries based on acceleration data.

The term “human function performance” is used to describe a range of observable factors which may be measured and quantified, thereby to assess the performance of one or more human attributes. Specific examples include:
Brain function performance testing. This is in some embodiments performed using various forms of testing hardware, and example of which being virtual reality (VR) based systems. An example VR-based system is described in detail further below. Such forms of function performance testing may test aspects of brain function including memory, vestibular and/or oculomotor performance. In some embodiments, brain function performance testing is performed thereby to assess impairment to brain function which may be associated with a detected head impact event.
Gait performance testing. This is preferably directed to assessing a subject's gate (for example while walking or running) by comparison to a benchmark/standard, which may be objectively defined for a population, or personalised for that subject. In some embodiments gait performance is measured using the mouthguard's sensors. Gate function is impaired where a subject's running/walking gate deviates from a defined “normal” range.
Cardiovascular performance testing, for example based on heart rate data, oxygen saturation, and the like. This is in some embodiments performed using sensors provided by the mouthguard. For example, Australian Patent Application 2021900584 described technology whereby circulatory system parameters are able to be measured via an instrumented mouthguard device.
Other various forms of clinical testing, such as EEG, ECG, detection/quantification of eye and/or eyelid movements (for example via infrared reflectance oculography spectacles), and the like.

Example embodiments include methods for assessing a brain injury, including:
(i) Accessing a first data set representative of an observed traumatic event, wherein the first data set is generated in response to data derived from one or more subject-worn motion sensors. For example, the first data set may be representative of a head impact event detected and quantified via an instrumented mouthguard device.
(ii) Accessing a second data set representative of human performance parameters (for example neurological conditions) following the observed traumatic event. The second data set is generated in response to data derived from subject performance data based on data derived from performance testing, for example via a brain function performance assessment delivered by a computer system.
(iii) Accessing a third data set representative of one or more biomarkers identified in a biological fluid harvested from the human subject after the traumatic event. For example, this may include collecting a blood and/or saliva sample. In some embodiments the instrumented mouthguard device is configured to collect one or more saliva samples at a time or times triggered in response to detection of an above-threshold head impact (as detected via one or more of the motion sensors), or in response to a software trigger (which may stem from a data processing algorithm and/or a manual trigger event). In other embodiments the saliva sample is collected my means other than he mouthguard.
(iii) Processing a combination of data from the first data set, the second data set and the third data set, thereby to define a fourth data set representative of an enhanced brain injury assessment.

In some embodiments, the fourth data set includes a metric representative of a deviation between any one or more of the following:
expected performance in the brain function performance assessment based on the observed traumatic event; (ii) actual performance in the brain function performance assessment based on the observed traumatic event.
expected biomarker presence (for example based on detection of salivary mRNA and/or ncRNA levels) based on the observed traumatic event; (ii) actual biomarker presence.
expected biomarker presence (for example based on detection of salivary mRNA and/or ncRNA levels) based on the performance in the brain function performance assessment; (ii) actual biomarker presence.
expected performance in the brain function performance assessment based on the observed biomarkers; (ii) actual performance in the brain function performance assessment.

In this manner, the distinct assessment metrics are used to identify relationships between anticipated and actual measurements. The three categories of assessment are based on differing principles (mechanism, performance observation, and biochemistry), and the interplay between these reveals additional detail regarding the way in which a specific individual has reacted to a particular impact event.

Expected performance may be derived using an AI classifier model, which is trained based on labelled data collected from historical observations.

The fourth data set is in some embodiments used to assess potential effectiveness of an intervention measure on a relationship between a traumatic event having defined characteristics and an effect on brain function performance assessment metrics. For example, the intervention measure may include medical treatments and/or nutritional supplements. In some embodiments, the fourth data set is used to assess potential effectiveness of an intervention measure on comparison between a current and historical relationship between a traumatic event having defined characteristics and an effect on brain function performance assessment metrics.

The fourth data set is also optionally used for other purposes, including (but not limited to: assessing potential compounding effects of multiple traumatic events over time; assessing athletic performance against a benchmark based on susceptibility to have reduced brain function performance in response to traumatic events during contact sports; assessing whether a brain injury is more or less serious than predicted, wherein the prediction is based on analysis of the first data set; and assessing whether brain function performance is better or worse than predicted, wherein the prediction is based on analysis of the first data set.

In some embodiments, correlations between head impact attributes (from an instrumented mouthguard device) and performance data (derived from human performance testing) are used as a basis to train an AI classifier (for example via a k-nearest-neighbours algorithm, or the like, which may be used to solve classification and/or regression problems). For example, data sets representative of head impact attributes are each labelled with associated correlated human function performance data. By way of example, in one embodiment the labelling occurs as follows:
A set of head impact data is collected.
A brain function test, in the form of a short-term memory test, is performed within a defined time period of collection of the set of head impact data.
The set of head impact data is processed into a form which provides various attributes (for example peak acceleration, direction of acceleration, and so on), which is optionally simplified via Principal Component Analysis or another such technique.
A label is defined for the results of the brain function test (in this example being in the form of a short-term memory test,). This is in some embodiments a label defined by a deviation from baseline expected function (preferably for the individual). For instance, the label may define a percentage of baseline performance (e.g. “80% performance”).
The label is applied to the processed set of head impact data.
The labelled data is used to train the classifier.
The classifier is tested, refined (e.g. by adjusting algorithmic parameter values), and validated.

It will be appreciated that labels may additionally/alternately be defined for other facets of brain performance (for example based on performance in other forms of memory test, oculomotor tests, vestibular system tests, and the like), and/or for factors other than brain function may be defined (such as % change in heart rate attributes against a baseline, a % change in gait against standard, and so on).

Outputs of the classifier provide predictions for the way in which an observed head impact (or impacts) will affect a human subject in terms of physical performance attributes. Such predictions are then optionally compared with actual observations, thereby to assess whether a given impact or impacts has had a greater/lesser/average effect on a subject. This provides useful insights into a range of factors, such as: (i) whether individual and/or cumulative factors are causing greater impairment than would be expected; and (ii) whether interventional measures, such as treatments and/or pharmaceutical measures and/or nutritional measures are having an effect on the functional effects of head impacts.

The classifier method above may also be performed in respect of labelling biomarker data based on brain function test results (for example thereby to assist in establishing correlations between forms of cognitive impairment and particular salivary biomarkers).

Examples described further below focus particularly on human performance assessment relating to brain performance, for example cognitive, memory, oculomotor and/or vestibular assessments. These are examples only, and a range of other data-driven human performance assessments may be used.

### Instrumented Mouthguard Technology Example

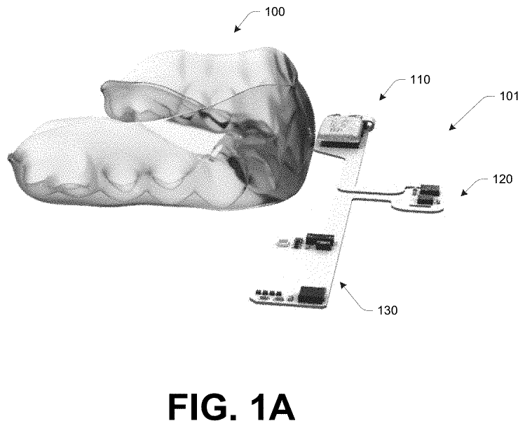

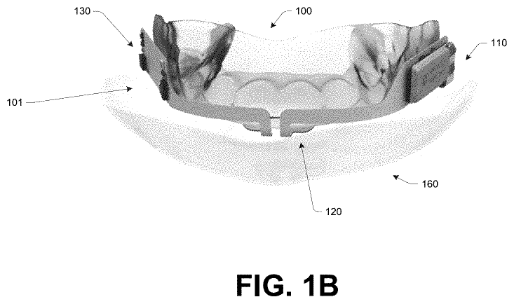

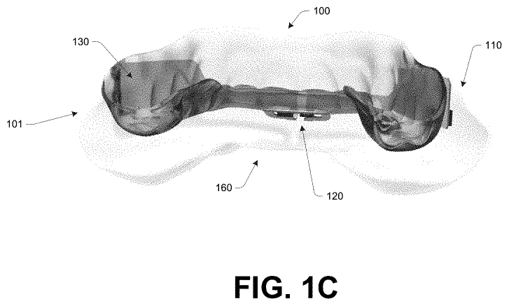

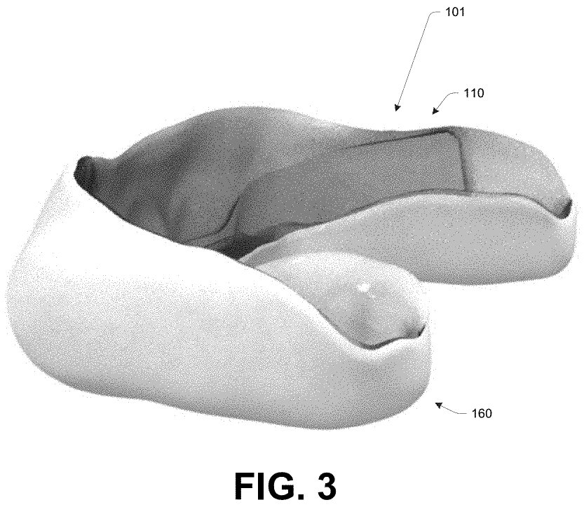

FIG. 1A to FIG. 10 illustrate an instrumented mouthguard device according to one embodiment. This example instrumented mouthguard is configurable to operate as a Head Impact Detection (HID) device, to provide both impact detection functionality and physical performance functionality. FIG. 3 provides a further example view.

The mouthguard comprises a mouthguard inner body 100, an instrumented component 101, and an outer mouthguard body 160. In the present embodiment the mouthguard inner body is custom formed based for a user based on a dentition scanning process, such that the mouthguard inner body provides a customised specifically to that user. The instrumented component 101 is then affixed to the inner body, and the outer body 160 sealed to the inner body 100 thereby to sandwich the instrumented component.

Additional detail regarding example instrumented mouthguard construction processes are provided in Australian provisional patent application 2020904214, entitled “multi-layered instrumented mouthguard devices, and methods for manufacturing of instrumented mouthguard devices”. The disclosure of that application is hereby incorporated by cross reference.

Instrumented component 101 includes a plurality of component zones 110, 120 and 130, which are spaced apart on a flexible PCB which follows a meandering path (i.e. the distance between component zones along the PCB is greater than the direct distance between the component zones).

The meandering path allows for mounting of the flexible circuit board substrate to the mouthguard inner body, such that the component zones are located in a frontal region of the mouthguard body (component zone 120); a side region of the mouthguard inner body (component zone 110); and an opposite side region of the mouthguard inner body from the second component zone (component zone 130). The frontal region is located on an opposite side of a teeth-receiving protective channel to the side region and opposite side region. In this example the frontal region is located on an inner side of the body relative to the protective channel, and the side region and opposite side regions are located on an outer side of the body relative to the protective channel. Outer body member cover 160 is mounted to the body thereby to seal components mounted on both the outer side of the inner body relative to the protective channel thereby to cover and the inner side of the inner body relative to the protective channel.

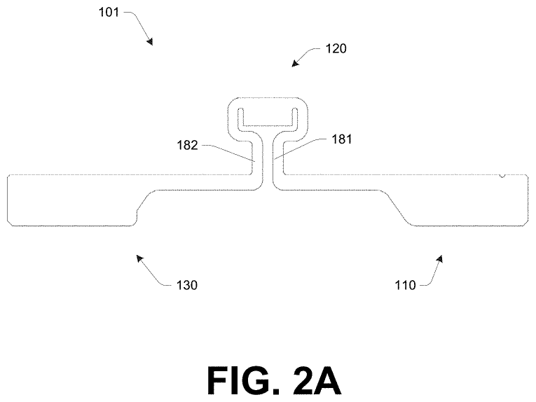

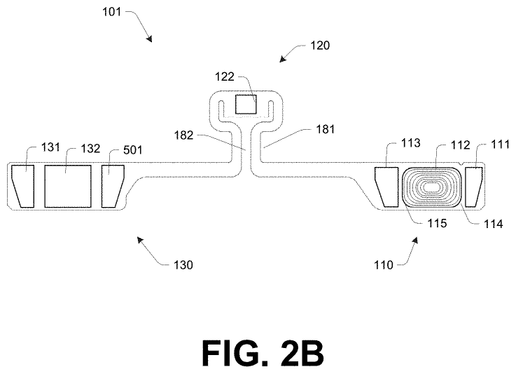

FIG. 2A and FIG. 2B illustrates an instrumented component 101 according to a further embodiment, this being configured for mounting in a mouthguard body thereby to provide an instrumented mouthguard.

As shown in FIG. 2A, component 101 is defined by a flexible circuit board substrate which is configured such that one or more conductive members electronically couples component zones (e.g. printed circuit board regions). The flexible circuit board in this manner defines a conductive member which is irregularly shaped such that it is configured to enable fitting of the component zones at desired locations on mouthguard bodies of varied shapes and sizes. More particularly, a PCB is formed to meander between component zones in a manner that allows for customisable fitting, whilst providing for added flexibility and robustness when the mouthguard is used. This presents a significant advantage over non-meandering PCBs, or the use of wires interconnecting distinct PCBs.

The PCB substrate illustrated in FIG. 2A may be of variable thickness, and/or have rigidity supports applied, thereby to adjust rigidity on a special basis thereby to protect PCB components as required for robustness.

Component 101 includes three component zones:
A right side component zone 110. In some implementations the right side component zone is configured to support PCB components including an accelerometer (3-axis), wireless communications unit, memory and microprocessor.
A frontal component zone 120. In some implementations, component zone 120 is split provides an accelerometer supporting zone configured to be positioned on the outer side of the front teeth (for a 3-axis accelerometer).
A left side component zone 130. In some implementations the left side component zone provides mounting locations for an accelerometer (3-axis), battery charging unit, and a battery mounting location.
The positioning of components described above, and shown in FIG. 2B, is an example only, and in other embodiments alternate configurations of components are distributed between the component zones.

A flexible connector member, defined by part of the PCB substrate onto which conductors connects these zones, has a first segment 181 which electronically couples right size component zone 110 and frontal component zone 120, and a second segment 182 which electronically couples front component zone 120 and left side component zone 130. As shown in FIGS. 2A and 2B, these segments are meandering. In this example, as with examples above, the meandering is such that, segment 181 is greater than the length of the separation of connection points with zones 110 and 120, and segment 182 is greater than the separation of connection points with zones 120 and 130.

The flexible connector member provides a flexible substrate onto which conductive strips and a plurality of PCB components are mounted (for example PCB components in zones 110, 120 and 130). In some embodiments the flexible substrate has an increased thickness in certain regions thereby to provide increased rigidity for PCB components that are susceptible to damage as a result of PCB flexion (for example see regions 111, 112 and 113 discussed below). In some embodiments additional materials are applied to the flexible substrate thereby to increase rigidity where required.

In the embodiment of FIG. 2B, zone 110 is defined by three substantially rigid PCB regions 111, 112 and 113, interconnected by comparatively flexible regions (flex connectors) 114 and 115. This enables a better fit of zone 110 to a curved surface; in the present embodiment it is configured to mounted in a right cheek region of the mouthguard body. Zone 110 includes a range of electronic components, including:
A 3-axis accelerometer.
A microprocessor (for example a Qualcomm CSR1012).
A memory module (for example a Macronix MX25L3233).
A wireless communications module, in this embodiment being a Bluetooth module coupled to a Bluetooth antenna (not shown), for example, an antenna configured to be mounted such that it runs across a frontal region of the mouthguard forward of a wearer's teeth.
A coupling port to a programming tab (not shown).
A Light-Emitting Diode configured to be visi/ble through the mouthguard body (not shown), in order to provide a device state indication to a user. For example, this is configured to be positioned behind the wearer's top lip.

It should be appreciated that the variations in rigidity within zone 110 (and across the component generally) is selected based at least in part of PCB components that are to be mounted at the various locations. For example, in one embodiment one or more of regions 111, 112 and 113 is not rigid, thereby to allow improved curvature upon application to the mouthguard body, and PCB components mounted to the non-rigid region are selected and/or mounted in such a manner to remain robust in spite to flexion in the PCB substrate.

Zone 120 includes a PCB region 122 including a 3-axis accelerometer (which is configured to be mounted to the mouthguard body in a location that in use is positioned behind front teeth).

Zone 130 is configured to be mounted on a left cheek region of the mouthguard body, and includes a PCB that carries a 3-axis accelerometer 131, along with a charging coil 132 to enable wireless charging of a battery unit 151.

In other implementations the battery unit is located in zone 110 or zone 120. In further embodiments additional components including the likes of gyroscopes may also be present at one or more of the component zones (for example a gyroscope in combination with an accelerometer at each component zone.

Segment 181 of the conductive member is configured such that, upon mounting to the mouthguard body, it traverses across a bottom region of the mouthguard body at a region approximately adjacent cuspid and first bicuspid (or, alternately, first and second teeth). This allows zone 120 to be provided on an internal region (behind teeth) and zone 110 provided on an external region (in front of teeth). A sealing cover is mounted to the body thereby to seal components mounted on both the outer side of the body relative to the protective channel thereby to cover and the inner side of the body relative to the protective channel.

In a further embodiment, component 101 or a variant thereof is embedded into a post-manufacture customised (e.g. a “boil and bite”) mouthguard. In such an embodiment, a standard generic form is injection moulded, and a user heats the mouthguard into a temporarily deformable state and bites firmly into it thereby to shape the resilient materials substantially to their teeth before it cools and becomes stable in the new customised shape.

In some embodiments the mouthguard device includes a component configured to collect a saliva sample at one or more defined times. For example, the defined time or times may be determined based on a trigger event, which occurs in the case that one or more of the motion sensor components within the mouthguard register motion (e.g. acceleration) satisfying an above-threshold condition. This preferably include collecting a first sample immediately following observation of the above-threshold condition, and a second sample after a predefined period of time has elapsed (thereby to enable analysis of short-term variations in salivary biomarkers in response to a head impact). This may be achieved via a sensor which is environmentally protected via a shield, with that shield being dissolvable subject to application of current/voltage from the mouthguard battery. A mechanically moving gate may also be used, for example a moving component which is magnetically actuated.

### Example HID Technology Framework

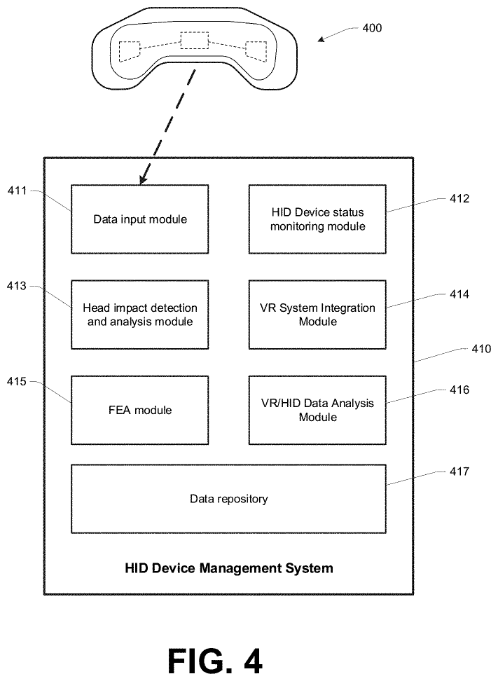

FIG. 4 illustrates an example HID technology framework, configured to enable monitoring of head impacts and physical performance for one or more subjects in a sporting activity.

The framework is described by reference to an HID device, in the form of a instrumented mouthguard 400, and an HID Device Management System 410, which takes the form of a computing device (for example a PC, notebook, tablet or smartphone) or a plurality of computing devices (for example various processing functionalities may be performed by cloud-hosted components). Instrumented mouthguard 400 includes a microprocessor configured to execute onboard software instructions, and it will be appreciated that various functions described as being performed by system 410 may in further embodiments be performed in whole or in part by mouthguard 400.

Software is described herein by reference to various modules. The term “module” refers to a software component that is logically separable (a computer program), or a hardware component. The module of the embodiment refers to not only a module in the computer program but also a module in a hardware configuration. The discussion of the embodiment also serves as the discussion of computer programs for causing the modules to function (including a program that causes a computer to execute each step, a program that causes the computer to function as means, and a program that causes the computer to implement each function), and as the discussion of a system and a method. For convenience of explanation, the phrases “stores information,” “causes information to be stored,” and other phrases equivalent thereto are used. If the embodiment is a computer program, these phrases are intended to express “causes a memory device to store information” or “controls a memory device to cause the memory device to store information.” The modules may correspond to the functions in a one-to-one correspondence. In a software implementation, one module may form one program or multiple modules may form one program. One module may form multiple programs. Multiple modules may be executed by a single computer. A single module may be executed by multiple computers in a distributed environment or a parallel environment. One module may include another module. In the discussion that follows, the term “connection” refers to not only a physical connection but also a logical connection (such as an exchange of data, instructions, and data reference relationship). The term “predetermined” means that something is decided in advance of a process of interest. The term “predetermined” is thus intended to refer to something that is decided in advance of a process of interest in the embodiment. Even after a process in the embodiment has started, the term “predetermined” refers to something that is decided in advance of a process of interest depending on a condition or a status of the embodiment at the present point of time or depending on a condition or status heretofore continuing down to the present point of time. If “predetermined values” are plural, the predetermined values may be different from each other, or two or more of the predetermined values (including all the values) may be equal to each other. A statement that “if A, B is to be performed” is intended to mean “that it is determined whether something is A, and that if something is determined as A, an action B is to be carried out”. The statement becomes meaningless if the determination as to whether something is A is not performed.

The term “system” refers to an arrangement where multiple computers, hardware configurations, and devices are interconnected via a communication network (including a one-to-one communication connection). The term “system”, and the term “device”, also refer to an arrangement that includes a single computer, a hardware configuration, and a device. The system does not include a social system that is a social “arrangement” formulated by humans.

At each process performed by a module, or at one of the processes performed by a module, information as a process target is read from a memory device, the information is then processed, and the process results are written onto the memory device. A description related to the reading of the information from the memory device prior to the process and the writing of the processed information onto the memory device subsequent to the process may be omitted as appropriate. The memory devices may include a hard disk, a random-access memory (RAM), an external storage medium, a memory device connected via a communication network, and a ledger within a CPU (Central Processing Unit).

In the example of FIG. 4, instrumented mouthguard 400 communicates with system 410 via a wireless connection. This may include a range of wireless technologies, including WiFi, Bluetooth, and/or other radio bands. In some embodiments communications between mouthguard 400 and system 410 progresses via one or more intermediate devices, including on-body retransmitting devices, devices in mesh networks, routers, and so on.

Data transmitted by mouthguard 400 is received by a data input module 411. Data input module 411 is configured to extract and sort input data, thereby to organise that data into memory accessible to system 410 (for example in one or more databases). This includes identifying a unique device identifier associated with mouthguard 400, which is preferably associated with a unique human subject. The data may include, for example, any one or more of: (i) a time-series of sensor readings, with associated time correlation data (such as a timestamp at the commencement of the series, and a known sampling rate); (ii) data packets representative of identified potential impact events (for example where the mouthguard is configured to operate in at least one setting where it transmits sensor data only where that sensor data has threshold values which indicate a potential impact); and (iii) output data from an onboard processing module (for example on onboard FEA module which provides an output based on a dosage input, the dosage input being derived from sensor data); and (iv) regular beacon/heartbeat data packets representative of device status. Other data may also be received, for example physiological data (such as heart rate, breathing rate, etc).

Data received and processed via input module 411 is stored in a data repository 417, where it is available for accessing and processing by other modules of system 410.

A HID device status monitoring module 412 is configured to process data received via input module 411 thereby to determine a current status of mouthguard 400 and optionally one or more further mouthguard devices. This may be used to assess whether one or more mouthguards are in a fault state or the like. In some embodiments module 412 is configured to enable two-way communication with mouthguard 400, for example to enable remote switching of mouthguard 400 between multiple distinct operational settings (for example one optimised for impact detection, and one optimised for physical performance assessment).

A head impact detection and analysis module 413 is configured to process data derived from sensors of mouthguard 400 thereby to provide metrics representative of severity of an observed impact event. It will be appreciated that there are a range of technologies which may be used for this processing, for example using techniques to process linear and/or rotational acceleration, optionally using AI methods and/or benchmarking against existing data. In this example, module 413 operates in conjunction with a Finite Element Analysis (FEA) module 415. Module 413 is configured to process sensor data thereby to define a dosage input signal. This may include processing time correlated data from multiple sensors thereby to determine an acceleration value at a defined location (for example at the centre of gravity of the subject's head, preferably based on transforms which are individually customised for the particular human subject based on their mouthguard and physical head configuration). This acceleration value is passed to FEA module 415, which performs analysis thereby to provide one or more metrics representative of predicted effect of the acceleration to the subject's brain, thereby to provide data which assists in understanding anticipated severity of a head impact.

A VR system integration module 414 is configured to interact with a VR system that delivers tests as described further above. In some embodiments this includes providing instructions thereby to control delivery of tests. In some embodiments this includes receiving outputs representative of testing results. A VR/HID analysis module is configured to combine data derived from mouthguard 400 and from the VR system testing in relation to one or more impact events.

### Subject Brain Performance Assessment via VR Technology

Some embodiments make use of performance assessment including brain performance assessment, via delivery of tests using virtual reality hardware.

As used herein, the term “virtual reality hardware” (VR hardware) is used to describe a wearable computer device which includes a display screen and motion sensors such as Inertial Monitoring Units (IMUs), which enable a user to observe a virtual three-dimensional space via head movements. The virtual reality hardware also includes one or more input devices, for example handheld controllers, triggers, buttons, microphones, and the like. In some cases additional peripheral stimuli devices may be included, for example a balance board device that is configured to deliver stimuli to upset a user's balance (relevant for vestibular system testing). In some embodiments VR hardware is controlled via a connected computer system, including one or more local and/or networked computer systems. These are used to control delivery of tests via the VR system, and for the collection and/or analysis of test results.

It is assumed for the purposes described herein that the VR hardware does not provide eye tracking functionality. Eye tracking functionality is used by a some known cognitive assessment technology platforms. However, such technology is complex, expensive, and often unreliable. As such, technology described herein has been adapted to operate without a need for eye tracking. In further embodiments the present technology may optionally be combined into a technology system that provides eye tracking.

In some embodiments, assessment of neurological performance includes delivering a sequence of tests of different test classes. By delivering such a sequence and in doing so transitioning between different test of different classes, the sequence causes an increase in cognitive loading to a subject. Variations in performance of tests in particular classes observed as a result of this increase in cognitive loading is in some embodiments additionally used to assess cognitive function and provide an indication of potential brain injuries.

In terms of tests that are applied, the technology makes use of a plurality of neurological tests that are renderable via hardware including a virtual reality system, with the plurality of neurological tests includes neurological tests belonging to a plurality of distinct test classes. In a preferred embodiment the test classes include a combination of the following:
Short-term memory tests.
Long-term memory tests.
Vestibular system tests. In some embodiments these make use of a peripheral balance board which is configured to measure variations of subject weighting in forward/backward/left/right directions in response to stimuli delivered via the VR hardware screen which is intended to upset the subjects balance For example
Reaction time tests. These may include ocular reaction time tests, whereby a user is presented with a visual stimulus and instructed to interact with an input device upon identifying that visual stimulus.
Executive cognitive function tests.

In some embodiments, a neurological assessment includes presenting a sequence of tests belonging to different classes (with adjacent tests having distinct classes) in a cyclical manner, and identifying variations in performance attributable to increasing of cognitive loading. This optionally includes comparing subject performance with a plurality of tests belonging to a particular one of the distinct test classes which are delivered non-adjacently with respect to the sequence.

### Example VR Testing Framework

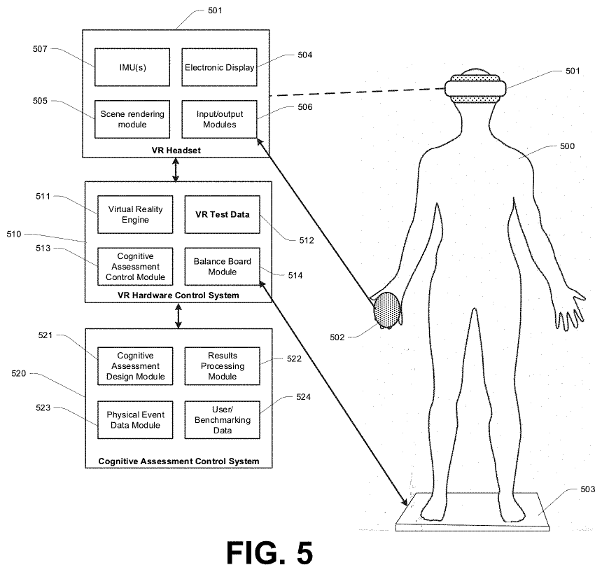

FIG. 5 illustrates a technology framework configured to enable assessment of a brain injury or other physiological condition. For example, in some embodiments this technology framework is used as a means to assess brain injuries for participants in a content sport, for example as a “sideline” assessment tool (although described as “sideline”, it will be appreciated that the assessment would usually be administered in an indoor space proximal a sporting field).

The framework of FIG. 5 is configured to perform a neurological assessment of a human subject 500. Subject 500 wears a VR headset 501, which is optionally a commercially available “off the shelf” system (for example an Oculus Rift/Quest/Go, HTC Vive, PlayStation VR, or the like; or alternately a VR system which makes use of a smartphone or the like in a specialised housing). Headset 501 includes one or more Inertial Measurement Units (IMUs) 507, thereby to enable observation of movement and control over scene rendering in response, based on operation of a scene rendering module 505 which causes rendering of a 3D VR scene on an electronic display 504. Input/output modules 506 are configured to deliver additional output stimuli to subject 500 (for example auditory, haptic, lights, and so on) and receive inputs (for example input via a handheld input device 502).

VR Headset 501 is coupled to a computer system 510, which executes computer code via one or more processors thereby to control operation of headset 510. In some embodiments some or all functions of computer system 510 are embedded into the headset data.

System 510 (and other components in FIG. 5) is described by reference to various modules. The term “module” refers to a software component that is logically separable (a computer program), or a hardware component. The module of the embodiment refers to not only a module in the computer program but also a module in a hardware configuration. The discussion of the embodiment also serves as the discussion of computer programs for causing the modules to function (including a program that causes a computer to execute each step, a program that causes the computer to function as means, and a program that causes the computer to implement each function), and as the discussion of a system and a method. For convenience of explanation, the phrases “stores information,” “causes information to be stored,” and other phrases equivalent thereto are used. If the embodiment is a computer program, these phrases are intended to express “causes a memory device to store information” or “controls a memory device to cause the memory device to store information.” The modules may correspond to the functions in a one-to-one correspondence. In a software implementation, one module may form one program or multiple modules may form one program. One module may form multiple programs. Multiple modules may be executed by a single computer. A single module may be executed by multiple computers in a distributed environment or a parallel environment. One module may include another module. In the discussion that follows, the term “connection” refers to not only a physical connection but also a logical connection (such as an exchange of data, instructions, and data reference relationship). The term “predetermined” means that something is decided in advance of a process of interest. The term “predetermined” is thus intended to refer to something that is decided in advance of a process of interest in the embodiment. Even after a process in the embodiment has started, the term “predetermined” refers to something that is decided in advance of a process of interest depending on a condition or a status of the embodiment at the present point of time or depending on a condition or status heretofore continuing down to the present point of time. If “predetermined values” are plural, the predetermined values may be different from each other, or two or more of the predetermined values (including all the values) may be equal to each other. A statement that “if A, B is to be performed” is intended to mean “that it is determined whether something is A, and that if something is determined as A, an action B is to be carried out”. The statement becomes meaningless if the determination as to whether something is A is not performed.

The term “system” refers to an arrangement where multiple computers, hardware configurations, and devices are interconnected via a communication network (including a one-to-one communication connection). The term “system”, and the term “device”, also refer to an arrangement that includes a single computer, a hardware configuration, and a device. The system does not include a social system that is a social “arrangement” formulated by humans.

At each process performed by a module, or at one of the processes performed by a module, information as a process target is read from a memory device, the information is then processed, and the process results are written onto the memory device. A description related to the reading of the information from the memory device prior to the process and the writing of the processed information onto the memory device subsequent to the process may be omitted as appropriate. The memory devices may include a hard disk, a random-access memory (RAM), an external storage medium, a memory device connected via a communication network, and a ledger within a CPU (Central Processing Unit).

System 511 includes a virtual reality engine 511, which is configured to process predefined VR content and cause that to be rendered via scene rendering module 505. In the present embodiment, the VR content includes a series of interactive tests provided in VR test data 512. Data 512 includes code for enabling execution of a plurality of neurological tests, including neurological tests belonging to a plurality of distinct test classes. Additional detail regarding these tests and test classes is provided further below. A cognitive assessment control module 513 is configured to enable selection and execution of the tests, for example based on a one-by-

### Example Testing Protocol

An example testing protocol is described below. It should be appreciated that this is an example only, and that various modifications can be made whilst remaining within the scope of the present invention.

The example testing protocol includes three high level categories:
Memory. This includes a test type for each of immediate memory, a test for working memory, and a test for delayed memory.
Oculomotor behaviour. This includes a visual assessment test, and a visual reaction test.
Vestibular. This includes a further visual reaction test, an acoustic reaction speed test, and a motor system posture control test.

Context to these tests, and examples of how the tests are implemented via the VR system are described in the following sections.

### Memory Testing—Context

One of the most common symptoms after concussion are memory impairments and attention deficits (Chen et al. 2004; McAllister et al. 1999, 2006). Even in asymptomatic patients, memory deficits can be found and are indicative of a concussion (Broglio et al. 2007).

Although in most patient these impairments resolve within the first few days after injury incident, in some cases they can become a long-term problem (Belanger et al. 2010; Gardner et al. 2010; Lovell et al. 2003; Reddy & Collins 2009). There is increasing evidence, that exposure to recurrent head impacts and concussions increases the likelihood for persistent memory impairments and other cognitive deficits (Amen et al. 2016; de Beaumont et al. 2009; Hume et al. 2017; Koerte et al. 2016b,a; Stamm et al. 2015; Wilde et al. 2016; Wright et al. 2016).

Many neuroimaging studies have been performed with the sophisticated aim to identify structural abnormalities that might explain chronic memory deficits. Cortical thinning is observed in retired professional athletes with memory dysfunctions and history of concussions (Koerte et al. 2016b). Frequently, changes in white matter are associated with memory impairments and reduced processing speed in concussed professional athletes (Bazarian et al. 2012; Wilde et al. 2016). There is some evidence, that often damaged brain regions in NFL players experiencing memory loss show abnormally low blood flow (Amen et al. 2016). Hippocampal atrophy and volume loss have been observed in TBI and are correlated with cognitive impairments including memory (Himanen et al. 2005; Strain et al. 2015).

Some studies aim to correlate abnormalities in brain activation pattern to memory impairments (Functional et al. 2013). It has been shown that that activation pattern of working memory in mild TBI patients differs from the control group (McAllister et al. 1999). Another interesting study found that those memory related activation pattern became comparable to those observed in control groups once symptoms have been resolved (Chen et al. 2004).

Despite abundance studies the mechanisms and factors contributing to transient and chronic memory impairments are poorly understood.

To improve current assessment methods by deciphering the effects of concussion on memory, the first step is understanding the mechanisms of memory formation and maintenance. There have been several models suggested to describe memory; however, the most influential model suggested over 50 years ago by Atkinson and Shiffrin is the multi-store or modal model (Atkinson & Shiffrin 1968). This model proposes that human memory has three separate components, namely the sensory memory, the short-term memory, and the long-term memory. All information we receive —consciously or subconsciously—enters our awareness through the sensory systems, e.g. visual or auditory system, and stays there for a short time of several hundred milliseconds. Once we pay attention, we can store this information for a short period of several seconds in our short-term memory. After roughly 15 to 30 seconds the information is forgotten through decay or displacement unless we can keep maintaining it actively through recall. Eventually, repeated rehearsal can transfer this information to the long-term memory, where we can retrieval the information after days, months and even years.

Later in 1974, Baddeley's model of working memory attempts to describe short-term memory more accurately by subdividing it into three further components (Baddeley & Hitch 1974). The Central executive is supervising the information flow from and to its two slave systems, namely the Visuospatial sketchpad, and the Phonological loop.

The Central executive acts as a supervisory system through directing focus and target information, this way making sure that short-term memory is actively working and can interact with long-term memory. It encodes, updates, and deletes information, structures information, controls attention and changes strategies in a task bound manner. This way, the Central executive controls the flow of information from and to its two lower systems, the Phonological loop, and the Visuo-spatial sketchpad.

The phonological loop (or “articulatory loop”) deals with sound or phonological information. This means even visually presented information can be articulated silently and encoded into the phonological storage. The visuo-spatial sketchpad keeps visual information, enables to create, revisit, and manipulate mental images.

As we can see in these very simplified memory models, memory formation is a process that depends on several factors including sensory input and attention. Therefore, while finding the cause of memory deficits can be challenging, the assessment of memory is highly sensitive to cognitive misfunctioning. Because of the complexity of factors contributing to and divers processing pathways involved in memory formation, it is likely that cognitive impairments are detected by memory tests. For instance, if an athlete has attention deficits and cannot take up new information—maybe because of slowed down processing times—it is logical that his short-term memory performance shows deficits.

### Memory Testing—Example Testing Protocol

Memory aspects of the example testing protocol are configured to test three aspects of memory, the immediate memory, working memory and delayed memory. The tests are described as Memory Test 1, Memory Test 2, Memory Test 3, and Memory Test 4.

In a Memory Test 1, the user interface the VR system is controlled to present a list of words for a defined period of time. Next, the user interface is controlled such that the participant is exposed to listed or decoy words one by one, and asked whether that word was present in the original list.

In Memory Test 2, the presentation of listed and decoy words is repeated (with the same list). This two tasks, where short-term memory is tested within the range of seconds to minutes, induce a learning process: The participant has time to take up information and through rehearsal memory is trained.

In Memory Test 3, which is delivered by the user interface of the VR system after performing the rest of the assessments (oculomotor and vestibular), the delayed memory is tested by recalling the same word list as per Memory Test 1 and Memory Test 2, without repeated exposure to the list (again optionally using listed/decoy word classification as a testing means).

Memory Test 4 aims to test the working memory. Five to nine nodes are shown, with one saying ‘START’ and another one saying ‘END’. A pattern connecting the nodes is drawn by the computer from start to end and disappears after a few seconds and the participant has to redraw the pattern. To ensure that the participant is not guessing (as opposed to utilising working memory), each pattern task is ended as soon as a wrong node is connected.

### Oculomotor Behaviour Testing—Context

Assessment of oculomotor behaviour plays a major role in diagnosing neurological abnormalities because of its complex neurocircuitry; functional neuroimaging has shown association of neuronal dysfunction with oculomotor performance (Bedell and Stevenson 2013; Johnson, Zhang, et al. 2015).

Oculomotor behaviour is divided into three eye movement categories: fixation, smooth pursuit, and saccades where we distinguish between vertical and horizontal saccades (Land and Tatler 2012). For fixation, the eye position is kept relatively still to focus a stationary image on the fovea, the central area on the retina with the highest sharp vision. Smooth pursuit is the targeting process of a moving stimulus to stabilize the image on the fovea. The saccades are rapid reflex-like eye movements between at least two fixation points. The different types of eye movement are associated with activation of different brain region, e.g., certain cortical areas control timing and location of saccades while cerebellar structures regulate saccade size and accuracy (Wong 2008). Further, stimuli direction activates different brain regions for saccades: For instance, horizontal saccades are initiated by the paramedian pontine reticular formation in the pons that receives inputs from the frontal eye field (FEF). For vertical saccades, the FEF signals to the rostral interstitial nucleus of the medial longitudinal fasciculus in the midbrain.

Saccades are usually characterized by velocity, duration, accuracy, and initiation time. We can test voluntary or self-paced saccades, i.e., saccades made between two stationary stimuli in a fixed time window. Traumatic brain injuries including concussion can affect eye movement like saccadic behaviour in humans. In individual TBI patients, the saccadic impairments have been associated with the degree of their head injury (Ventura et al. 2015, 2016).

In concussion assessment, balance and cognitive abilities are tested, without exploiting the potential of eye movement assessment: Common tests such as SCAT5 ask about the ability of the patient to open their eyes and blurred vision while visual deficits are not assessed in detail. Symptom reports are unreliable, subjective, and quick cognitive assessments alone as memory tests in SCAT5 can fail to detect a range of cognitive deficits. Therefore, it is recommended in several studies to include assessment of eye movements in concussion assessment, e.g., via the King-Devick (K-D) test, which is a rapid visual performance measure (Mucha et al. 2014). It examines reading speed and language production and has a short assessment of saccade but does not test other eye movements. There are other tests like Vestibular/Ocular Motor Screening (VOMS) assessing amongst other saccades, smooth pursuits, and fixations (Ventura, Balcer, and Galetta 2014). However, the VOMS underlies subjective human bias and has not come into general use in sport-related concussion assessment yet. Emphasizing that vision uses half the pathways in the brain, it has great potential for concussion assessment (Heitger 2003; Heitger et al. 2009; Ventura et al. 2014). Roughly 65-90% of concussed patients reveal oculomotor disruptions, slowed saccades and deficits in smooth pursuit (Cochrane et al. 2019). A study in 2019 showed that saccadic and visual reaction times are significantly lower in concussed individuals (Hunfalvay et al. 2019, 2020).

Furthermore, recent studies showed that horizontal and vertical saccades can be used as diagnostic marker for TBI (Cochrane et al. 2019; Heick and Bay 2021; Hunfalvay et al. 2019, 2020; Stuart et al. 2020). The saccadic velocity, accuracy and targeting was measured in healthy controls and TBI patients of different TBI severity. The performance on targeting alone could distinguish between patients with mild and severe TBI and healthy controls. In alignment with previous studies, it was shown that horizontal saccades and targeting are most sensitive to distinguish between the different test groups.

With progressing technology, many devices, that can track eye movements, and several evaluation metrices have been developed (Cochrane et al. 2019). However, the influence of cognitive and visual functions on eye-movement has not been considered. Moreover, the validity and reliability of those tools and the processing of eye tracking data is insufficiently reported. The analysis methods as the tools are yet in a development stage. One study even reports that tests were repeated until a valid trial was completed (Andersson et al. 2010).

A good overview over the many different available eye movement instruments is provided in a critical review by Stuart et al. (2020) (see table below). Most studies used infra-red eye tracking devices in a seated position, a few while standing or walking. Sampling frequencies of 50-200 Hz were reported for saccadic eye movement detection, while sampling rates of 60 Hz are sufficient to detect saccades (DiCesare et al. 2017; Johnson, Zhang, et al. 2015; Johnson, Hallett, and Slobounov 2015), a higher frequency should be considered to detect all features or deficiencies of eye movements (DiCesare et al. 2017; Johnson, Zhang, et al. 2015; Johnson, Hallett, et al. 2015). Another weakness of those eye movement instruments that needs to be considered is the vulnerability of the technology to head and body movements and calibration errors.

Optimization of the tools (reliability and validity) and standardization of analysis metrices needs to be further progressed until eye-tracking devices can be used as standard concussion assessment aids.

### Oculomotor Behaviour Testing—Example Testing Protocol

Common (computerized) neuropsychological test batteries focus on cognitive aspects and do not include assessment of vision and saccades. For the present of the example testing protocol described herein, we include additionally to the cognitive performance a hand-eye coordination test and a vision test that challenges the horizontal smooth pursuit (in a preferred embodiment taking in 2 minutes).

The example testing protocol includes a test (OBT Test 1) which challenges those eye movements identified to be significantly affected in concussion without a need for eye tracking hardware. A discussed above, horizontal saccades, smooth pursuit and targeting are most sensitive to distinguish between controls and concussed patients. In the example testing protocol, a visual assessment test is delivered via the VR hardware in which:
The subject is presented with a graphic showing a ball bouncing between two walls.
The subject is instructed to track the ball only by moving eyes with a fixed had position.
The subject is instructed to provide a designated input every time the ball hits the wall.

The software logic underlying the test is configured to cause the difficulty slowly over the two minutes (e.g. by increasing ball velocity and changes in direction). Changes in velocity and direction are randomised to challenge the subject's predicting abilities and attention. Performance is evaluated by the accuracy of task performance. In case that a subject experiences abnormality in the oculomotor functioning, such as previously mentioned slowed saccades, troubles targeting for smooth pursuit, this is reflected in overall performance metrics (i.e. accuracy in providing inputs).

The example testing protocol also includes a test (OBT Test 2) which assesses hand-eye coordination and visual reaction, amongst other factors: The subject is presented with a virtual environment in which a game is presented. The game includes objects being thrown (or otherwise travelling through the air) towards the subject. The subject is instructed to catch the objects. Here, increase the difficulty levels is achieved by changes in ball velocity and number of balls in the game. The test is administered with the subject in a seated position.

### Vestibular Testing—Context

Humans have a set of vestibular organs on each side of the head, directly behind and interconnected to the acoustic system. The vestibular system detects the position and movement of the head and contributes this way most significantly to the sense of balance. It helps to coordinate movements of head, eyes, and our body posture. This coordination is an automated process that of which a person is unaware. However, if this system is malfunctioning, it can lead to many different symptoms such as sickness, motions sickness, vertigo, dizziness, nausea, and uncontrollable eye movements, which are all commonly reported symptoms after a concussion (Bear, Connors, and Paradiso n.d.; Calzolari et al. 2021; Echemendia et al. 2017).

The vestibular system is interconnected with several functions, e.g., our sight including visual processing pathways. Thus, damage to a broad range of brain areas such as the inner ear, nerve, brainstem, cerebellum, and cerebral hemispheres could all affect vestibular functioning. Therefor a multi-level assessment of balance and vestibular related eye-movements is required to screed those functions.

The vestibular system, like the acoustic system the cochlea, uses hair cells to translate movements. These hair cells are in interconnected in fluid filled chambers, the vestibular labyrinth. It consists of two structures, the otolith organs and the semicircular canals. The former detects the force of gravity and acceleration such as tilts of head. This way, we naturally sense where “up” and “down” is. The semicircular canals sense head rotations and angular accelerations. These canals are orientated in three directions, in approximately orthogonal planes i.e., 90° between any two of them. By this means, they can detect different kind of movement and acceleration, each in a different direction.

The vestibular system does not only help to control head, eye, and body position with the information about gravity and acceleration, it uses at the same time feedback from other body parts to finetune this information. The vestibular system receives itself inputs from the brain including the cerebellum (part of hindbrain) like visual, acoustic and the motor system, which all contribute to our sense of balance to maintain posture. For example, it is interconnected with spinal motor neurons, that control the leg muscles. Hereby, we can maintain balance even on a dynamic surface or object, such as on a surfboard. Another important function of the vestibular system is to maintain our visual focus on a moving point or when the body is moving, called the vestibulo-ocular reflex. This reflex triggered system can directly control the eye muscles meaning the vestibular system is connected to our oculomotor functions.

The assessment of balance in TBI and concussion is a relatively important factor for return to play decisions (link to intro chapter). In some cases, patients even develop chronic imbalance problems (Hoffer, Balough, and Gottshall 2007). A recent study indicated that 62% of TBI patients show dysfunctional balance performance while half of them did not report any balance problems which indicates the necessity of balance assessment in concussion management (Marcus et al. 2019). The mechanisms behind balance dysfunction and vestibular function in patients with TBI is not yet well understood. Concussion studies addressing this question are still evolving (Calzolari et al. 2021).

There is evidence that the cause for imbalance in a majority of acute TBI patients lies in vestibular related dysfunctions (Marcus et al. 2019; Sargeant et al. 2018). Interestingly, patients with dysfunctional vestibular system, perform better walking than standing (Brandt, Strupp, and Benson 1999; Calzolari et al. 2021).

Vestibular—Example Testing Protocol

In the example testing protocol, tests are implemented to cover various different functions that contribute to the sense of balance. Apart from the vestibular system, balance depends on visual and acoustic cues as mentioned above. In addition, it has been shown that dual tasks in balance are more effective to detect balance impairments (Beauchet and Berrut 2006; Camicioli et al. 1997; Howell, Buckley, et al. 2018; Howell, Kirkwood, et al. 2018).

In view is this, the sample testing protocol has three tests (Vestibular Test 1, Vestibular Test 2 and Vestibular Test 3) performed in a standing position on the balance board. The system is configured to collect data about postural stability during different conditions and tasks.

Vestibular Test 1 is a repeat of OBT Test 1 (or optionally OBT Test 2), but in a standing position. Gaze control and saccades are less accurate in a standing position than sitting position (Boulanger, Giraudet, and Faubert 2017; Rougier and Garin 2007). By this approach, the system is able to determine whether standing affects results of the visual reaction test.

Vestibular Test 2 relates to acoustic reaction speed. The vestibular and auditory system are connected at an early processing stage because the organism is exposed constantly to moving sound sources from the environment while moving itself. Auditory inputs can improve postural stability, these two senses influence each other. The dorsal cochlear nucleus where auditory nerve fibres form their first synapses, integrates auditory and vestibular information (Rougier and Garin 2007; Stevens et al. 2017; Wigderson, Nelken, and Yarom 2016). Interestingly, patients with vertigo compensate the feeling of imbalance by focusing more on visual cues and are highly visually dependent (Bronstein 1995). Surprisingly, they show better adaptation to visual disorientations (Guerraz et al. 2001). Because our vision too contributes to postural stability, and there is a slight chance that patients can adapt to the feeling of imbalance by stronger visual dependency, the auditory reaction test is performed in a dark room in the VR environment (Boulanger et al. 2017; Rougier and Garin 2007).

In Vestibular Test 3, the balance board itself is used here as a controller. This task is performed by instructing the subject to engage in a process of shifting weight from one foot to the other, forward, and backward, i.e., the centre of pressure or the centre of gravity on the board is moved. This aims to challenge control of the motor system that controls posture, and provides a test that required precise control of posture:

Integration of Brain Function Performance Assessments with Instrumented Wearable Technologies

In some embodiments, human function performance assessment technology (such as VR-based cognitive assessment as described in detail further above, and/or other forms of human function assessment such as gait, heart rate, and other clinical observations) is used to assess physical cognitive function in combination with data derived from physically observing a traumatic event (such as a head impact). This observation may be achieved via a worn device having motion sensors, for example an instrumented mouthguard device, instrumented helmet device, or the like.

In this context, the term “instrumented” denotes that a wearable item, such as a mouthguard, helmet or the like, has electronic components integrated therein or thereon, for example motion sensing components such as accelerometers, gyroscopes, and the like. An example of an instrumented mouthguard device is provided further below as context, however it will be appreciated that technology described herein may be implemented with a range of instrumented wearable devices. For the purposes of the following description, an instrumented mouthguard is used as an example only.

For the purposes of these embodiments, the use of VR assessment technology is used as a primary example of “brain function performance assessment”. However, other forms of tests may be used, including a range of cognitive, vestibular and ocular-motor tests, these being delivered via VR technologies or other forms of technology, thereby to deliver objective data outcomes which are representative of one or more metrics of brain function performance.

In overview, various embodiments described herein bring together brain function performance testing and wearable sensor data, thereby to provide an enhanced assessment of brain injuries. For the purposes of the present description, this is referred to as “enhanced brain injury assessment”, as it takes into consideration a combination of the mechanism of injury (based on sensor data), and physical effects observed following the injury (from physical testing).

Enhanced brain injury assessment, may be used for a range of purposes, including:
Assessing whether one or more attributes of physical performance are affected to a greater or lesser degree than expected compared with benchmark data.
Assessing whether one or more attributes of physical performance are affected to a greater or lesser degree than expected, for a given subject, compared with benchmark data for a sample population (for example a population segment selected with similar demographic and/or other characteristics relative to the subject). For example, using simplified metrics, assume that for a given subject on a given day, sensor data shows an impact rated as a 5/10. For the sample population, this level of impact on average results in a performance metric of 80/100. Then, for the subject, the actual performance metric is measured at 60/100. This provides insights into how the subject responds to traumatic brain events relative to the relevant sample population, and can assist in treatment or the like.
Assessing compounding effects of successive brain traumatic events, for example by changes in relationships between mechanism of injury data and brain function performance data. For example, in the case of a specific subject, there may be an observed variance between a metric representative of mechanism of injury (determined from sensor data) and a metric representative of brain function performance (from brain function performance assessment data) over time. For example, using simplified metrics, assume that for a given subject on a given day, sensor data shows an impact rated as a 5/10, and a brain function performance metric of 80/100. Then, on another day in the future, sensor data shows an impact rated as a 5/10, and a brain function performance metric of 60/100. This demonstrates that a similar level of injury has had a greater effect on the same metric of performance, indicating that there has been a change in the way in which the subject's brain responds to trauma. By way of a wider data set, such observations may be made based on predictive benchmarking even where the impacts are of different ratings (as there can be a predicted relationship between impact rating and performance metric).
Assessing the effects of immediate-term intervention measures following a traumatic brain event. In these embodiments, brain function performance assessments are conducted periodically following a traumatic event in combination with implementation of an intervention measure, and rates of performance improvements (or otherwise) are assessed against benchmark data thereby to assess whether (and the extent to which) intervention measures have an effect on recovery. For example, intervention measures may include medications, meditation, rest, or the like. This form of enhanced brain injury assessment is useful in both research into treatment of brain injuries, and in assessing effectiveness of treatment of brain injuries. For example, using simplified metrics, assume that for a given subject on a given day, sensor data shows an impact rated as a 5/10, and a brain function performance metric of 80/100. Assume a benchmark recovery for the subject, based on personal and/or demographic historical data, shows a predicted return to a metric of 90/100 in 12 hours and 100/100 in 36 hours. In this case that actual assessment in presence of an intervention measure shows an actual return to a metric of 100/100 in 12 hours, there is an indication that the intervention measure may have been effective. This may also be used in the context of improving athletic performance, for example in assessing the effectiveness of treatment regimes (medical or otherwise) in reducing the immediate effect on performance associated with brain trauma (including mild trauma during sport), thereby to assist in athletic performance during contact sports.
Assessing the effects of longer-term intervention measures following a traumatic brain event. In these embodiments, brain function performance assessments are conducted following successive traumatic event in combination with implementation of an intervention, and rates of performance improvements (or otherwise) are assessed against benchmark data thereby to assess whether (and the extent to which) intervention measures have an effect on recovery. For example, intervention measures may include medications, meditation, rest, or the like. This form of enhanced brain injury assessment is useful in both research into treatment of brain injuries, and in assessing effectiveness of treatment of brain injuries. This may be used, by way of example, to assess the extent to which a treatment program is successful in reducing compounding brain injury effects over the course of a longer period, for example over the course of a sporting career. For example, using simplified metrics, assume that for a given subject on a given day, sensor data shows that a subject has experienced between 10-20 impact rated as greater than 6/10 on average for the past 5 years. Benchmark data might indicate that, at present, there would have been a 30% decrease in brain function performance metrics in response to a given level of impact rating over that time. However, the subject might have no decrease in 30% decrease in brain function performance metrics in response to a given level of impact rating over that time. This is an indication that the intervention measure may have been effective.

In the examples above, numerical identifiers for impact ratings and brain function performance metrics are for explanation only. In practice, an impact rating can be any metric or combination of metrics derived from sensor data collected during a traumatic event, for example derived from rotational and/or linear acceleration, and/or computer models (for example FEA models) which use sensor data (or data derived from sensor data) as input. A brain function performance metric may be derived from a brain function performance having a single test type (for example a balance test), or from a combination of multiple test types (for example as described further above). These may be delivered via VR and/or other means.

One class of embodiment provides a method for assessing a brain injury which combines data from an instrumented wearable device, such as an instrumented mouthguard, with data derived from a VR-based assessment. Such an embodiment may take the form of a method including:
(i) accessing a first data set representative of an observed traumatic event, wherein the first data set is generated in response to data derived from one or more subject-worn motion sensors;
(ii) accessing a second data set representative of neurological conditions following the observed traumatic event, wherein the second data set is generated in response to data derived from subject performance data in a neurological assessment delivered by a virtual reality system; and
(iii) processing a combination of data from the first data set and the second data set thereby to define a fourth data set representative of an enhanced brain injury assessment.

The first data set in some embodiments includes a metric derived from processing of data provided by the instrumented mouthguard device (for example a metric representing severity of trauma, preferably derived from measurement of rotational acceleration of the subject's head). In other embodiments the first data set includes output from a brain model that is executed based on the data derived from an instrumented mouthguard device (or another device providing one or more subject-worn motion sensors). This may include a brain model in the form of a Finite Element Analysis (FEA) model which makes use of head motion data (for example linear and/or rotational accelerations) thereby to model predicted effects on the internal structure of the brain.

In some embodiments, a processing method includes processing a combination of data from the first data set and the second data set by identifying a correlation between an output of the FEA model and performance in the neurological assessment. For example, this may include benchmarking against prior results for the same subject and/or different subjects. This optionally enables testing predictions of injury severity and effect on subject function, leading to a better understanding of the effects of a particular traumatic event.

The enhanced brain injury assessment may serve a range of purposes, including the following:
Improving knowledge with respect to relationships between observations in sensor data and observations from a neurological assessment. For example, this may include improving understanding of relationships between categories of impact (for example as defined in terms of linear acceleration, rotation acceleration, intensity, period, and the like) with observations of neurological condition.
Improving personalised head injury management, for example by tracking, with respect to an individual, a relationship between impact attributes and neurological effects. For example, this may include identifying whether a person experiences increased effects as a result of cumulative impacts (e.g. whether results in subsequent neurological assessments worsen over time in the event of impacts having similar or lesser intensity).
Enabling improved management of sporting activities. For example, a protocol may be implemented whereby participants in a sporting activity wear instrumented mouthguard devices, and data from these devices is monitored (for example the data is transmitted wirelessly to a computer system), with a greater than threshold intensity recording for a given participant (for example defined in terms of linear/rotation acceleration, and/or acceleration as a function of time) triggering a requirement that the participant undergo a VR-based assessment. A combination of the data from the instrumented mouthguard (either wirelessly transmitted data, or more detailed data downloaded following the incident) and results of the VR-based assessment are then used to influence next steps. For example, the next steps may include: whether the participant is permitted to return to the activity; whether the participant is permitted to return to the activity after a predefined rest period; whether the participant is prevented to return to the activity that day; and/or whether the participant is required to undergo a particular further medical examination. Determination of next steps may occur on the basis of a review of the data in combination with a review and judgement of a medical officer.
Identifying potential false-positive results from instrumented equipment. For example, in some implementations a reading of threshold intensity from an instrumented mouthguard during a sporting activity may trigger a compulsory VR-based assessment (and optionally assessment from a medical officer). In the event that the neurological assessment results reveal better subject performance than predicted based on the instrumented device data, this may provide a factor in favour of the subject returning to the sporting activity.

In the examples above, data from an instrumented mouthguard has been described by reference to sensor data, such as acceleration data. In some embodiments, the instrumented mouthguard data is data derived from a computerised model (for example an FEA model) which receives input data from the instrumented mouthguard (or other instrumented wearable device).

An example method is illustrated in FIG. 8A. In this method, block 801 represents commencement of monitoring of mouthguard data. This may include either or both of monitoring at a mouthguard device (for example where the device activates responsive to identification that it is being worn) and/or monitoring at a computer system which is wirelessly connected to a plurality of mouthguard devices.

Block 802 represents a process whereby mouthguard data indicates an event above a threshold intensity. This intensity may be measured based on one or more of the following factors: rotational acceleration peak; linear acceleration peak; rotational acceleration as a factor of time; linear acceleration as a factor of time; output from a computerised model such as an FEA model; and/or other metrics. The intensity may be determined based on inboard processing at the mouthguard device, based on processing of data transmitted from the mouthguard device to a computer system, or a combination of both. This may be used to trigger a process by which a participant is removed from participation in the sporting activity.

Block 803 represents generation of an alert representative of a requirement that a participant undergo a VR-based assessment. This is optionally triggered based on the event at block 802. The participant then undergoes the VR-based assessment at block 804.

Block 805 represents a process including combining data representative of performance in the VR assessment with data derived from the mouthguard. This combination of data is used for one or more of the purposes described above, for example in terms of assisting decision making with respect to the participant in an outcome implementation process as represented by block 806 (e.g. return to activity or not, additional medical scans, etc) and/or adding to a data set which correlates impact data with cognitive performance data for a plurality of participants and/or for the individual participant (for example to allow benchmarking of future events and/or optimisation and improvement of data analysis processes).

A further class of embodiment provides a method for assessing a brain injury whereby a sensor data and VR-based assessment data are used in combination for the purposes of a brain injury assessment. A method according to this class includes:
(i) Accessing a first data set representative of an observed traumatic event, wherein the first data set is generated in response to data derived from one or more subject-worn motion sensors. For example, this may include data representative of accelerations during an event, and/or data from a computer model (for example a FEA model) which is derived in response to the motion sensors.
(ii) Based on the first data set, configuring a virtual reality system to deliver a neurological assessment having defined parameters to the subject. This, in some cases, includes customising the parameters of the neurological assessment based on the first data set, optionally in combination with personalised data for the subject. For example, these parameters may include the type of test(s), level of difficulty or cognitive loading, duration of test, and the like.
(iii) In response define a second data set representative of subject performance in the assessment; and performing a brain injury assessment based on a combination of the first data set and the second data set.

In this manner, data relating to a actual physical traumatic event may be used for either or both of: (i) influencing parameters of a VR-based cognitive assessment which is delivered to a subject; and (ii) providing an enhanced assessment which combines data derived from observation of the event with results of a VR neurological assessment.

The first data set in some embodiments includes a metric derived from processing of data provided by the instrumented mouthguard device (for example a metric representing severity of trauma, preferably derived from measurement of rotational acceleration of the subject's head). In other embodiments the first data set includes output from a brain model that is executed based on the data derived from an instrumented mouthguard device (or another device providing one or more subject-worn motion sensors). This may include a brain model in the form of a Finite Element Analysis (FEA) model which makes use of head motion data (for example linear and/or rotational accelerations) thereby to model predicted effects on the internal structure of the brain.

In some embodiments, a processing method includes processing a combination of data from the first data set and the second data set by identifying a correlation between an output of the FEA model and performance in the neurological assessment. For example, this may include benchmarking against prior results for the same subject and/or different subjects. This optionally enables testing predictions of injury severity and effect on subject function, leading to a better understanding of the effects of a particular traumatic event.

In embodiments where VR assessment derived data and trauma-derived data are combined, a process is optionally performed thereby to derive metric representative of a deviation between: (i) expected performance in the neurological assessment based on the observed traumatic event; and (ii) actual performance in the neurological assessment based on the observed traumatic event. This is optionally used to test and/or validate, via the second data set, a hypothesis as to the nature of a brain injury made based on the first data set.

The process of performing a brain injury assessment based on a combination of the first data set and the second data set may include any one or more of the following:
Comparing results of the VR-assessment with predicted/benchmarked results based on measured characteristics of the traumatic event, based on data previously collected across a plurality of subjects (e.g. whether the current subject experiencing greater or lesser cognitive impact than average for an impact of that intensity).
Comparing results of the VR-assessment with predicted/benchmarked results based on measured characteristics of the traumatic event, based on data previously collected for the current subject (e.g. whether the current subject experiencing greater or lesser cognitive impact than for a previous impact of that intensity).

It will be appreciated that bringing together data from both measurement of an event via wearable instrumented devices and subsequent VR-based cognitive assessment allows for a significantly enhanced overall assessment program. Understanding relationships between events themselves (for example in terms of acceleration types and the like) and cognitive effects is a powerful tool in understanding and treating brain injuries, including on an individually customised basis.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

It should be appreciated that in the above description of exemplary embodiments of the invention, various features of the invention are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A method for processing subject data related to a head impact event, the method including:
receiving input data derived from one or more observational data sources, including at least one of: (i) data derived from an instrumented mouthguard device associated with an impact event, wherein the instrumented mouthguard device is worn by the subject during the impact event; and (ii) data derived from a cognitive assessment performed by the subject within a predefined time of the impact event;
receiving a set of biomarker data collected from the subject within a predefined time of the impact event;
labelling the biomarker data with one or more metrics derived from the input data; and
providing the labelled biomarker data to a classifier module, thereby to train the classifier module to predict, in respect of a new set of biomarker data, values for the one or more metrics predicted to be associated with that biomarker data.

2. A method for processing subject data related to a head impact event, the method including:
receiving primary input data for a human subject derived from one or more observational data sources, including at least one of: (i) data derived from an instrumented mouthguard device associated with an impact event, wherein the instrumented mouthguard device is worn by the subject during the impact event; and (ii) data derived from a cognitive assessment performed by the subject within a predefined time of the impact event;
receiving secondary input data derived from processing of a biological substance extracted from the human subject; and
comparing the primary input data and the secondary input data, based on a data correlation protocol which associates relationships with predicted attributes, thereby to perform a head injury assessment.

3. A method for assessing a brain injury, the method including:
accessing a first data set representative of an observed traumatic event, wherein the first data set is generated in response to data derived from one or more subject-worn motion sensors, worn by a human subject during the traumatic event;
accessing a second data set representative of human performance following the observed traumatic event, wherein the second data set is generated in response to data derived from subject performance data in a human function performance assessment delivered by a computer system and undertaken by the human subject after the traumatic event;
accessing a third data set representative of one or more biomarkers identified in a biological fluid harvested from the human subject after the traumatic event;
processing a combination of data from the first data set, the second data and the third data set thereby to define a fourth data set representative of an enhanced brain injury assessment.

4. An instrumented mouthguard device including:
a plurality of motion sensing components;
a microprocessor; and
a saliva sensor component.

5. A device according to claim 4 wherein the saliva sensor component is triggered in response to processing of data received via at least one of the plurality of motion sensing components.

6. A device according to claim 4 wherein the saliva sensor component is configured to collect a plurality of saliva samples, and wherein the saliva sensor component is triggered to collect a first sample in response to processing of data received via at least one of the plurality of motion sensing components, and a second sample a predefined period following the first sample.
